(19) United States
(12) Patent Application Publication
(10) Pub. No.: US 2023/0125463 A1
(43) Pub. Date: Apr. 27, 2023

(54) Self-contouring plate for bone fractures

(71) Applicant: Yesser Nadim, Somerset, KY (US)

(72) Inventor: Yesser Nadim, Somerset, KY (US)

(21) Appl. No.: 17/508,813

(22) Filed: Oct. 22, 2021

(51) Int. Cl. A61B 17/80

(52) U.S. Cl. CPC A61B 17/8085; A61B 17/8014; A61B 17/8057; A61B 2017/00477

(57) ABSTRACT

The self-contouring plate for bone fractures allows a surgeon to bridge a bone fracture, primarily in bones of complex shape where the use of plates or screws is difficult. The self-contouring plate is formed from a series of similar or identical rigid elements, the elements able to bend and rotate with respect to each other. This flexibility is initially helpful as the surgeon contours the device to the shape of the bone. When the desired shape is reached, the elements are locked into place. The length of device is adjusted by adding or removing elements, much like a necklace. Each element of the self-contouring plate includes a ball that extends away from a body, a cavity for receiving the ball of the neighboring plate, and a screw to compress the ball within the cavity.

### FIELD

This invention relates to the field of treating bone fractures and more particularly to a device for treating a complex bone fracture.

### BACKGROUND

The treatment of complex bone fractures has moved beyond the antiquated treatments of full-body casts and traction.

Instead, the use of screws and plates helps surgeons to fix fractures in position, allowing the patient to regain partial mobility while the bone mends.

But the use of mechanical fracture supports, such as plates, is complicated by bones with complex shapes, such as the pelvis.

Current methods require the surgeon to contour, or bend, a plate during surgery, the plate intended to match the contour of the patient’s bone.

This contouring is difficult and imperfect, and can result in fractures that are only partially reduced. The imperfect contouring can cause loss of reduction obtained prior to applying the plate. This increases healing time and decreases patient mobility.

What is needed is a device that contours to the bone, the device then locked into shape and affixed to the bone.

### SUMMARY

The self-contouring plate for bone fractures allows a surgeon to bridge a bone fracture, primarily in bones of complex shape where the use of plates or screws is difficult.

The self-contouring plate is formed from a series of similar or identical rigid elements, the elements able to bend and rotate with respect to each other. This flexibility is initially helpful as the surgeon contours the device to the shape of the bone. When the desired shape is reached, the elements are locked into place.

The length of device is adjusted by adding or removing elements, much like a necklace. Each element of the self-contouring plate includes a ball that extends away from a body, a cavity for receiving the ball of the neighboring plate, and a screw to compress the ball within the cavity.

Each element can rotate in three directions – swivel left and right, or yaw; tilt forward and backward, or pitch; and rotate about its centerline, or roll. This freedom of rotation is created by the ball-and-socket connection that joins each element to the next. When the desired arrangement and angles are reached, the ball is squeezed with a cap screw, or compression screw, fixing the ball-and-socket joint in position. Restated, the ball-and-socket joint has both a locked position or condition, and an unlocked position or condition.

The ball-and-socket connection allows for a full range of motion. The preferred embodiment has the ability swivel in 45 degrees of yaw, tilt in between 45– and 90-degrees of pitch, and rotate in 360 degrees of roll.

One or more screw holes in each element allow placement of bone screws, fixing the device to the underlying bone.

The entire device is intended for permanent internal implantation, directly against the bone. The device does not protrude through muscle or skin, and does not have elements that remain external to the body.

The centerline of each element of the device is preferably consistent, with the centerline of the ball matching that of the centerline of the body. When installation is complete, there are no protruding elements that could cause discomfort by aggravating the surrounding tissues. Stated differently, in the preferred embodiment the thickness of the device is substantially consistent, without protruding elements.

The self-contouring plate is strengthened by being positioned against the surface of the bone. The plates and connections are directly against the surface of the bone, avoiding rotational moments that would increase the force against the plates. This is in contrast to the prior art devices, which were placed partially outside the patient’s skin, resulting in traumatic and uncomfortable pins that passed through the patient’s bone and muscle. The prior art placed the points of rotation away from the bone, thus requiring a thicker mechanism to compensate for the resulting rotational forces.

The self-contouring plate includes a solid ball, without a through-hole for a fixation screw. The result is a stronger ball connection with more material. The ball is preferably spherical, with the only interruption to its surface being the neck that connects the ball to the body of the plate.

Additionally, by using a solid ball, the greatest range of movement is possible. Requiring placement of a fastener through the ball limits angular rotation of the ball because the hole in the ball must line up with a second hole for receipt of the fastener.

This additional range of motion is helpful in complex fractures, such as fractures of the pelvis and acetabulum and fractures of the distal radius.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a top view of a single element of the self-contouring plate.

FIG. 2 illustrates a top view of multiple elements of the self-contouring plate.

FIG. 3 illustrates a view of the rotation of elements with respect to each other of the self-contouring plate.

FIG. 4 illustrates a side cross-sectional view of a single element of the self-contouring plate.

FIG. 5 illustrates a side cross-sectional view of multiple elements of the self-contouring plate.

FIG. 6 illustrates a first installed view of the self-contouring plate.

FIG. 7 illustrates a second installed of the self-contouring plate.

FIG. 8 illustrates an end-on cross-sectional view, before placement of the compression screw, of the self-contouring plate.

FIG. 9 illustrates an end-on cross-sectional view, after partial placement of the compression screw, of the self-contouring plate.

FIG. 10 illustrates an end-on cross-sectional view, after tightening of the compression screw, of the self-contouring plate.

FIG. 11 illustrates a detailed view of the socket connection of the self-contouring plate.

FIG. 12 illustrates a side cross-sectional view of a single element with a vertical compression screw.

FIG. 13 illustrates a side cross-sectional view of a single element with an angled compression screw.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

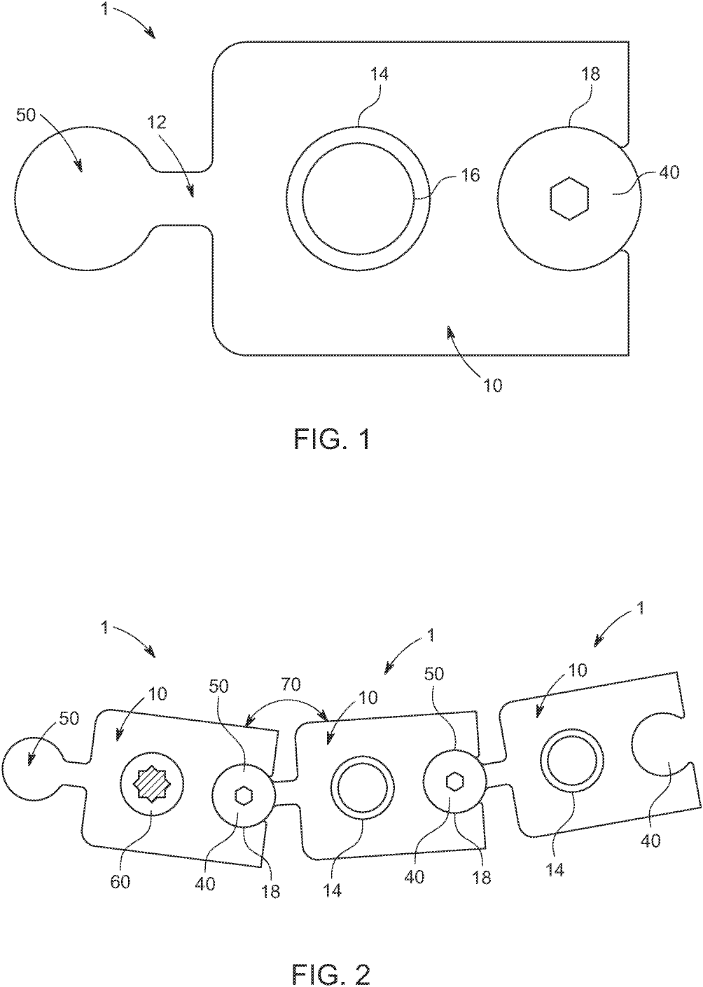

Referring to FIG. 1, a top view of a single element of the self-contouring plate is shown.

Each self-contouring plate 1 includes a body 10, tapering to a neck 12 followed by a head 50.

The body 10 includes a non-threaded hole 14 with optional chamfer 16 along the upper perimeter of the non-threaded hole 14. In alternative embodiments, the hole 14 is threaded.

The rear of the body 10 includes a socket 18 to receive a ball 50 of the next self-contouring plate 1.

A compression screw 40 acts to fix the position of the ball 50 within the socket 18 after placement of the self-contouring plate 1.

Referring to FIG. 2, a top view of multiple elements of the self-contouring plate is shown.

Multiple self-contouring plates 1 are affixed in a series, allowing the device to span a fracture.

The heads 50 are fixed within the sockets 18 of subsequent self-contouring plate 1.

Adjacent self-contouring plates 1 can be placed at angles with respect to each other, shown here as a swiveling left and right, or yawing 70.

Each body 10 is optionally fixed in place using a bone screw 60.

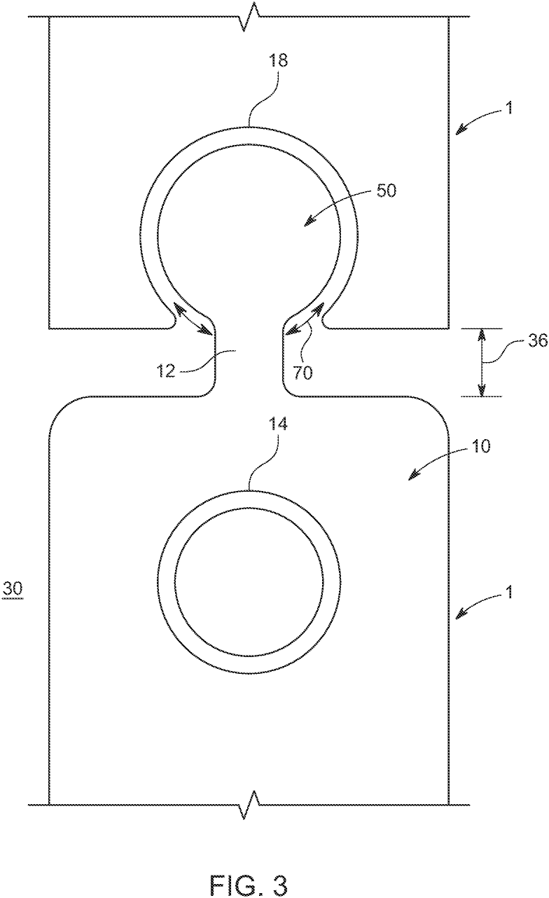

Referring to FIG. 3, a view of the rotation of elements with respect to each other is shown in horizontal plane 30.

The head 50 sits within the socket 18, with the self-contouring plates 1 able to rotate at the connection between the head 50 and the socket 18.

A linear gap 36 exists between the bodies 10 of the adjacent self-contouring plates 1. By varying the length of the neck, the linear gap 36 may be changed, which is one way of creating greater tolerance between adjacent self-contouring plates 1, and thus allowing for greater angular deviations.

Swiveling left and right is shown as yawing 70.

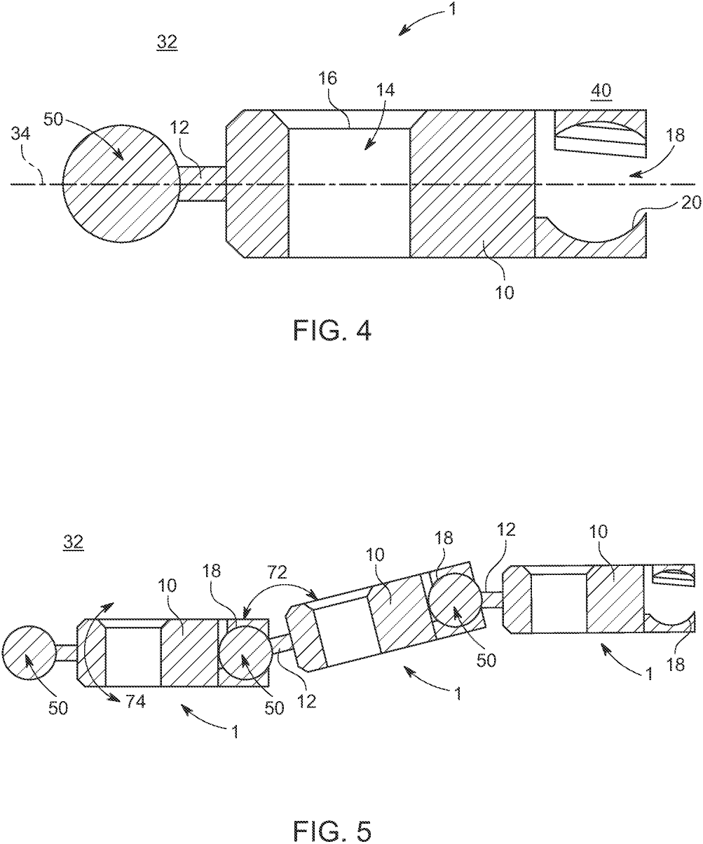

Referring to FIG. 4, a side cross-sectional view of a single element of the self-contouring plate is shown in vertical plane 32.

A single self-contouring plate 1 is shown in cross-section. The body 10 tapers to a neck 12, with head 50.

The hole 14 passes through the body 10, with optional chamfer 16.

The socket 18 includes a cup 20 that will support a head 50, and a compression screw 40 that will grip the head.

A centerline 34 passes through the self-contouring plate 1, splitting the head 50, neck 12, and body 10.

Referring to FIG. 5, a side cross-sectional view of multiple elements of the self-contouring plate is shown.

Multiple self-contouring plates 1 are shown connected in series, tilted with respect to each other. Tilting forward and backward is shown as pitching 72. Rotation about its centerline 34 (see FIG. 4) is shown as rolling 74.

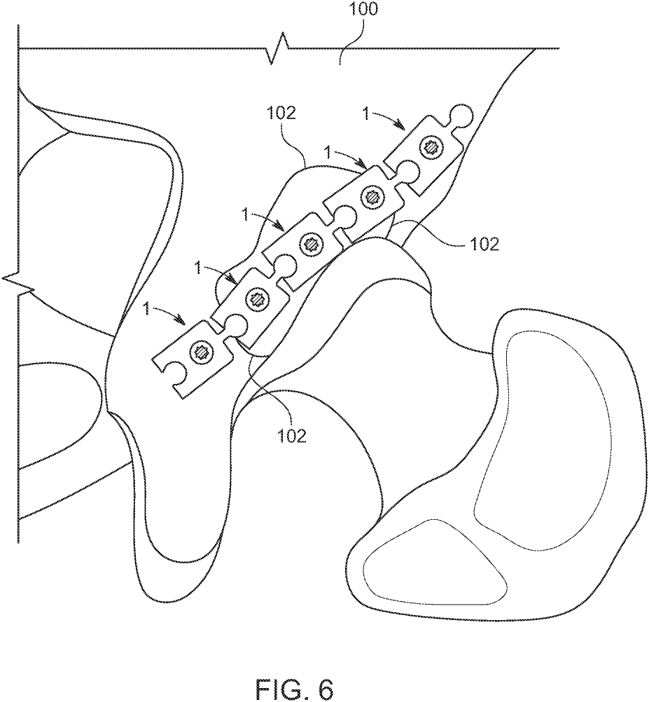

Referring to FIG. 6, an installed view of the self-contouring plate is shown.

A series of self-contouring plates 1 are connected to bridge a fracture line 102 in a pelvis 100. The compression screws 40 are omitted to better show the angled position of the plates 1 with respect to each other.

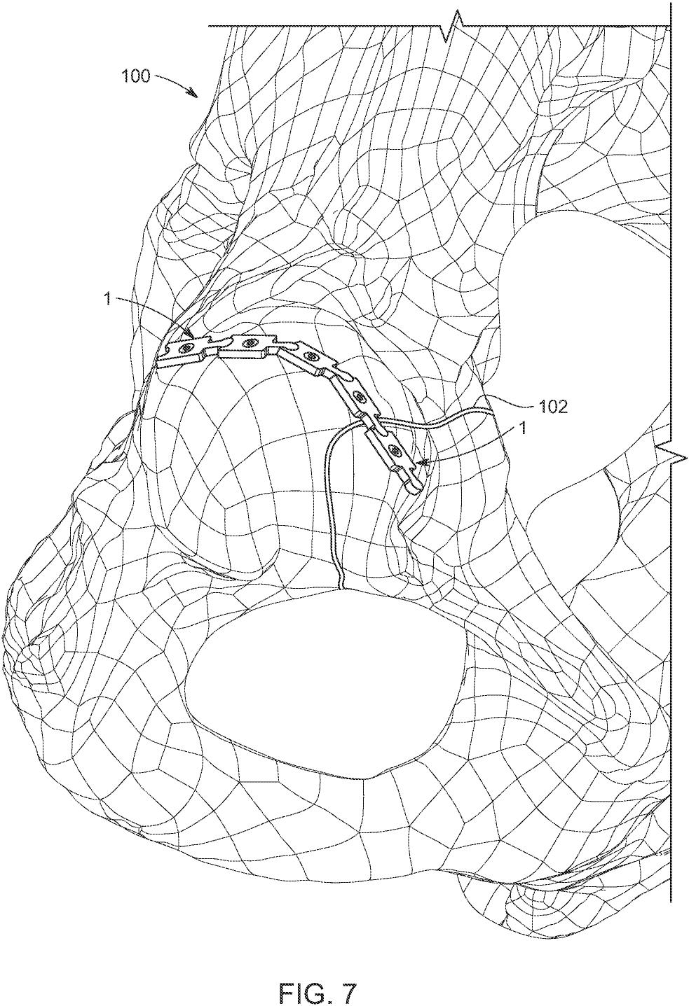

Referring to FIG. 7, a second installed of the self-contouring plate is shown.

Again shown is a series of self-contouring plates 1, connected to bridge a fracture line 102 in a pelvis 100. The pelvis 100 is curved, but the series of self-contouring plates 1 compensates, following the curvature.

The compression screws 40 are omitted to better show the angled position of the plates 1 with respect to each other.

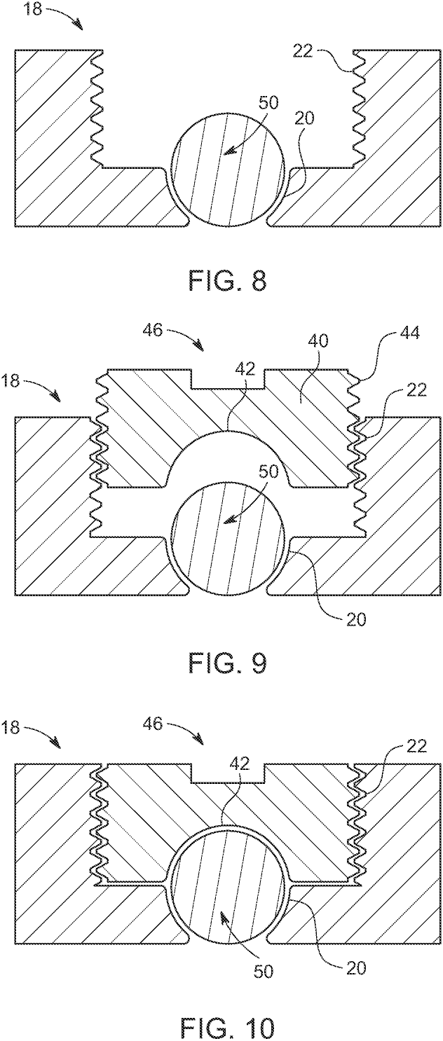

Referring to FIG. 8, an end-on cross-sectional view, before placement of the compression screw, of the self-contouring plate is shown.

The socket 18 includes a cup 20, in which a head 50 can sit.

The socket 18 includes threaded walls 22.

Referring to FIG. 9, an end-on cross-sectional view, after partial placement of the compression screw, of the self-contouring plate is shown.

The compression screw 40 has threads 44 that interface with the threaded walls 22 of the socket 18. The compression screw 40 includes an inverted cup 42 to grip the head 50, and a recess 46 allowing for insertion of a hex head screwdriver or other bit to control rotation of the compression screw 40.

Referring to FIG. 10, an end-on cross-sectional view, after tightening of the compression screw, of the self-contouring plate is shown.

The compression screw 40 is set in place, compressing the ball 50 into the cup 20, thus preventing further rotation.

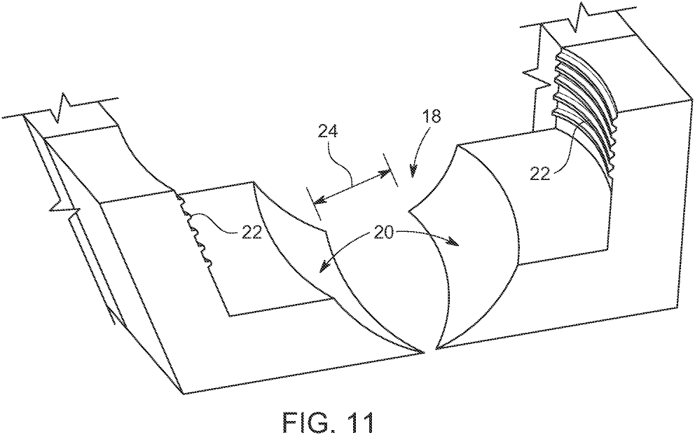

Referring to FIG. 11, a detailed view of the socket connection of the self-contouring plate is shown.

The socket 18 is shown with cup 20 and threaded walls 22. The cup is shown separated into two halves, creating a lower gap 24.

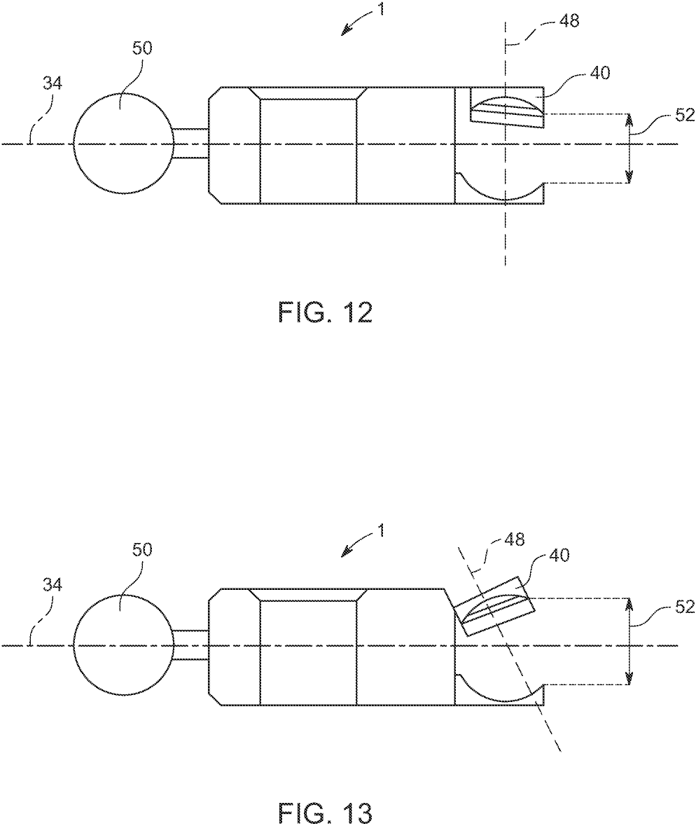

Referring to FIG. 12, a side cross-sectional view of a single element with a vertical compression screw of the self-contouring plate is shown.

The self-contouring plate 1 is shown with a compression screw 40, the compression screw centerline 48 set at ninety degrees with respect to the centerline 34.

Referring to FIG. 13, a side cross-sectional view of a single element with an angled compression screw of the self-contouring plate is shown.

The self-contouring plate 1 is shown with a compression screw 40, the compression screw centerline 48 set at less than ninety degrees with respect to the centerline 34. Specifically, the compression screw 40 is angled toward the head 50. The result is that the neck upward rotation gap 52 of the embodiment in FIG. 13 is greater than that in FIG. 12, thus allowing greater freedom for the self-contouring plate 1 to pitch with respect to its neighbor.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction, and arrangement of the components thereof without departing from the scope and spirit of the invention or without sacrificing all of its material advantages. The form herein before described being merely exemplary and explanatory embodiment thereof. It is the intention of the following claims to encompass and include such changes.

What is claimed is:

1. A device for implantation within a body, the device fully beneath a layer of skin, the device to bridge a bone fracture, the device comprising:
a first element and a second element linked by a ball-and-socket joint;
the ball-and-socket joint having an unlocked position and a locked position;
the ball-and-socket joint sharing a centerline with the first element and the second element, thus permitting a low-profile implantation against the bone fracture;
the ball-and-socket joint separated, with a ball on the first element and a socket within the second element;
the first element and the second element each including a hole for a bone screw;
whereby the ball-and-socket joint is initially movable for to allow the device to contour to a shape of the bone fracture, the device then stiffened by compression of the ball within the ball-and-socket joint.

2. The device for implantation within the body of claim 1, wherein the ball is spherical and solid, thus permitting 360-degree rotation of the first element with respect to the second element along the centerline.

3. The device for implantation within the body of claim 1, further comprising:
a compression screw;
the compression screw interfacing with the socket;
the compression screw including a first position corresponding with the unlocked position of the ball-and-socket joint, and a second position corresponding with the locked position of the ball-and-socket joint;
the compression screw causing the locked position of the ball-and-socket joint by pressing against an exterior surface of the ball, and not passing through the ball;
whereby the compression screw is actuated after placement of the device across the bone fracture, locking the device into a desired shape.

4. The device for implantation within the body of claim 3, wherein:
the socket includes threaded walls;
the compression screw includes external threads;
the compression screw interfaces with the external threads of the socket, thereby allowing a surgeon to compress the ball into the socket without passing a fastener through the ball.

5. The device for implantation within the body of claim 1, wherein:
the hole for the bone screw includes a chamfered upper perimeter, thus allowing the bone screw to be set flush with an upper surface of the first element.

6. The device for implantation within the body of claim 1, wherein:
the first element can roll 360 degrees along the centerline with respect to the second element;
the first element can swivel side-to-side at least 45 degrees with respect to the second element;
the first element can tilt up-and-down at least 45 degrees with respect to the second element.

7. The device for implantation within the body of claim 3, wherein:
the first element can roll 360 degrees along the centerline with respect to the second element;
the first element can swivel side-to-side at least 45 degrees with respect to the second element;
the first element can tilt up-and-down at least 45 degrees with respect to the second element.

8. A device to bridge a bone fracture of a curved bone, the device comprising:
a plurality of rigid elements;
each element of the plurality of rigid elements including:
a body;
the body including a hole for a bone screw;
a socket within a first end of the body;
a ball-shaped head at a second end of the body;
the ball-shaped head sharing a centerline with the body;
the ball-shaped head able to be movably interfaced with the socket of an adjacent rigid element of the plurality of rigid elements;
the ball-shaped head having an unlocked condition with respect to the socket, and the ball-shaped head having a locked condition with respect to the socket;
a neck;
the neck joining the body to the ball-shaped head;
the plurality of rigid elements joined by ball-shaped heads, allowing each element of the plurality of rigid elements to have six degrees of rotational freedom with respect to each other.

9. The device to bridge a bone fracture of a curved bone of claim 8, wherein the ball-shaped head is spherical and solid, thus permitting 360-degree rotation of the plurality of rigid elements with respect to each other.

10. The device to bridge a bone fracture of a curved bone of claim 8, further comprising:
a compression screw;
the compression screw interfacing with the socket;
the compression screw including a first position corresponding with the unlocked condition of the ball-shaped head within the socket, and a second position corresponding with the locked condition of the ball-shaped head within the socket;
the compression screw causing the locked condition of the ball-shaped head within the socket by pressing against an exterior surface of the ball-shaped head, and not passing through the ball-shaped head;
whereby the compression screw is actuated after placement of the device across the bone fracture, locking the device into a desired shape.

11. The device to bridge a bone fracture of a curved bone of claim 10, wherein:
the socket includes threaded walls;
the compression screw includes external threads;
the compression screw interfaces with the external threads of the socket, thereby allowing a surgeon to compress the ball-shaped head into the socket without passing a fastener through the ball-shaped head.

12. The device to bridge a bone fracture of a curved bone of claim 8, wherein:
the hole for the bone screw includes a chamfered upper perimeter, thus allowing the bone screw to be set flush with an upper surface of the body.

13. The device to bridge a bone fracture of a curved bone of claim 8, wherein:
each element of the plurality of rigid elements can roll 360 degrees along the centerline with respect to an adjacent element of the plurality of rigid elements;
each element of the plurality of rigid elements can swivel side-to-side at least 45 degrees with respect to an adjacent element of the plurality of rigid elements;
each element of the plurality of rigid elements can tilt up-and-down at least 45 degrees along the centerline with respect to an adjacent element of the plurality of rigid elements.

14. The device to bridge a bone fracture of a curved bone of claim 11, wherein:
each element of the plurality of rigid elements can roll 360 degrees along the centerline with respect to an adjacent element of the plurality of rigid elements;
each element of the plurality of rigid elements can swivel side-to-side at least 45 degrees with respect to an adjacent element of the plurality of rigid elements;
each element of the plurality of rigid elements can tilt up-and-down at least 45 degrees along the centerline with respect to an adjacent element of the plurality of rigid elements.

15. A device for stabilizing a fracture of a bone, the bone having a complex shape, the device comprising:
a first rigid element and a second rigid element joined by a flexible connection;
the first rigid element and the second rigid element each including a hole for a bone screw to be installed;
the flexible connection being a ball-and-socket joint;
the ball-and-socket joint including a ball and a socket;
the socket within the first rigid element;
the ball extending from the second rigid element;
the ball and the second rigid element having a common centerline;
the flexible connection having a first position that allows movement of the ball with respect to the socket, and a second position that locks the ball with respect to the socket;
a compression screw locking the ball with respect to the socket when in the second position;
the compression screw resting against an outside of the ball, and not passing through the ball;
the compression screw including cup-shaped recess that matches an outer surface of the ball;
whereby the device allows for stabilization of the fracture despite the complex shape of the bone.

16. The device for stabilizing a fracture of a bone of claim 15, wherein:
the compression screw causing a locked position of the ball-and-socket joint by pressing against an exterior surface of the ball, and not passing through the ball;
whereby the compression screw is actuated after placement of the device across the fracture, locking the device into a desired shape.

17. The device for stabilizing a fracture of a bone of claim 16, wherein:
the socket includes threaded walls;
the compression screw includes external threads;
the compression screw interfaces with the external threads of the socket, thereby allowing a surgeon to compress the ball into the socket without passing a fastener through the ball.

18. The device for stabilizing a fracture of a bone of claim 15, wherein:
the hole for the bone screw includes a chamfered upper perimeter, thus allowing the bone screw to be set flush with an upper surface of the first rigid element.

19. The device for stabilizing a fracture of a bone of claim 15, wherein:
the first rigid element can roll 360 degrees along the common centerline with respect to the second rigid element;
the first rigid element can swivel side-to-side at least 45 degrees with respect to the second rigid element;
the first rigid element can tilt up-and-down at least 45 degrees with respect to the second rigid element.

20. The device for stabilizing a fracture of a bone of claim 16, wherein:
the first rigid element can roll 360 degrees along the common centerline with respect to the second rigid element;
the first rigid element can swivel side-to-side at least 45 degrees with respect to the second rigid element;
the first rigid element can tilt up-and-down at least 45 degrees with respect to the second rigid element.
